(19) United States
(12) Patent Application Publication
(10) Pub. No.: US 2005/0020672 A1
(43) Pub. Date: Jan. 27, 2005

(54) Methods of extending mammalian life span via iron chelating compounds that reduce free radical damage in mammals

(75) Inventor: Donald Lynn Bissett, Hamilton, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/741,645

(22) Filed: Dec. 19, 2003

Related U.S. Application Data
(62) Division of application No. 09/552,305, filed on Apr. 19, 2000, now abandoned.
Application No. 09/552,305 is a continuation-in-part of application No. 08/224,949, filed on Apr. 8, 1994, now abandoned.

(51) Int. Cl. A61K 31/34; A61K 31/137

(52) U.S. Cl. 514/471; 514/651

(57) ABSTRACT

The subject invention involves a method of extending the life span of mammals by reducing free radical damage in mammals wherein the method comprises administering to a mammal a composition comprising a safe and effective amount of a compound selected from 1-phenyl-1,2-propanedione-2-oxime, benzoylacetone, piroctone, 2-furildioxime, 2-furilmonoxime, diethyldithiocarbamic acid, deferoxamine and 1,2-dimethyl-3-hydroxy-pyrid-4-one, or a pharmaceutically acceptable salt thereof, or mixtures of the subject compounds and/or their salts.

### CROSS REFERENCE TO RELATED APPLICATION

This application is a Divisional of application Ser. No. 09/552,305 filed Apr. 19, 2000 in the name of Bissett, which is a Continuation-In-Part of application Ser. No. 08/224,949 filed on Apr. 8, 1994 in the names of Bissett and Shaya.

### TECHNICAL FIELD

The subject invention relates to the field of protecting mammals from free radical damage and conditions accelerated by free radical damage. Specifically, the subject invention relates to novel methods of extending the average lifespan of mammals by using certain iron-chelating compounds that reduce the level of free radicals in mammalian cells.

### BACKGROUND OF THE INVENTION

Free radicals, in particular oxygen radicals, in mammalian cells arise from a variety of environmental sources. Such sources include smoke, pollution and radiation in addition to normal cell metabolism and inflammatory processes. Free radicals are known to be damaging to biological tissue. In the body, the potential for damage by free radicals is greatly increased by the presence of iron (and to a lesser extent by the presence of copper), which catalyzes their conversion to less stable and therefore more reactive radical species. The resulting effects of such radicals on components such as structural proteins, membrane lipids and nucleic acids include alteration or loss of tissue and cell function, cell death, and cancer. Based on a growing body of evidence, it is also believed that the accumulated damage from free radicals, including those produced as by-products in normal metabolism, is responsible for chronological aging. This has been termed the free radical theory of aging.

To combat the damaging effects of free radicals, especially oxygen radicals, free radical scavengers and anti-oxidants have been used. These compounds react with the radical species to convert them to stable, nonreactive materials.

It is an object of the subject invention to provide methods for extending the life span, in particular the average life span of mammals by reducing free radical damage in mammalian cells.

It is also an object of the subject invention to provide compositions useful for extending the life span, in particular the average life span of mammals by reducing free radical damage in mammalian cells.

### SUMMARY OF THE INVENTION

The subject invention involves a method of extending the life span, in particular the average life span of mammals by reducing free radical damage in cells of living mammals wherein said method comprises the step comprising administering to a mammal a composition comprising a safe and effective amount of a compound selected from 1-phenyl-1,2-propanedione-2-oxime, benzoylacetone, piroctone, 2-furildioxime, 2-furilmonoxime, diethyldithiocarbamic acid, deferoxamine and 1,2-dimethyl-3-hydroxy-pyrid-4-one, or a pharmaceutically acceptable salt thereof, or mixtures of the subject compounds and/or their salts.

### DETAILED DESCRIPTION OF THE INVENTION

It has been unexpectedly found that compositions comprising the subject compounds exhibit the ability to reduce levels of free radicals in mammals (e.g., humans, dogs, cats, mice, etc.). While the subject invention is not limited to any particular mode of action, it is believed that the subject compounds may reduce the level of free radicals in mammalian cells by preventing the formation of the most damaging radical species. The subject compounds are believed to bind to metals, in particular iron and copper and especially iron, such that the metals cannot participate in the formation of the above-mentioned radical species. The subject compounds, which act as metal chelators, in particular iron chelators, are unexpectedly effective against both environmentally induced radical production as well as the endogenous radical production arising from metabolism

As used herein, “alkyl” means a substituted or unsubstituted carbon-containing chain which may be straight or branched; saturated, monounsaturated (i.e., one double or triple bond in the chain), or polyunsaturated (i.e., two or more double bonds in the chain; two or more triple bonds in the chain; one or more double and one or more triple bonds in the chain).

As used herein, “topical application” means directly laying on or spreading on outer skin.

As used herein, “pharmaceutically-acceptable” means that salts, drugs, medicaments or inert ingredients which the term describes are suitable for use in contact with the tissues of humans and lower animals without undue toxicity, incompatibility, instability, irritation, allergic response, and the like, commensurate with a reasonable benefit/risk ratio.

As used herein, “safe and effective amount” means an amount of compound or composition sufficient to significantly induce a positive modification in the condition to be treated, but low enough to avoid serious side effects (at a reasonable benefit/risk ratio), within the scope of sound medical judgment. The safe and effective amount of the compound or composition will vary with the particular condition being treated, the age and physical condition of the patient being treated, the severity of the condition, the duration of the treatment, the nature of concurrent therapy, the specific compound or composition employed, the particular pharmaceutically-acceptable carrier utilized, and like factors within the knowledge and expertise of the attending physician.

As used herein, “free radical” means an atom or group of atoms that have one or more unpaired electrons. Such atoms or groups of atoms are highly reactive and unstable species. Also included among “free radicals” are other oxygen species which are highly reactive toward biological systems. Some “free radicals” are produced by the catalytic action of metals (e.g., iron or copper) or are more reactive toward biological systems in the presence of metals (e.g., iron or copper). Specific non-limiting examples of “free radicals” as defined above are:

| | |
|---|---|
| superoxide | O₂⁻ |
| hydroperoxyl radical | HO₂· |
| peroxide ion | O₂²⁻ |
| hydroperoxyl anion | HO₂⁻ |
| hydroxyl radical | HO· |
| singlet oxygen | ¹O₂ |
| hydrogen peroxide | H₂O₂ |
| ferryl iron | FeO²⁺ |
| perferryl iron | FeO₄³⁻ |

As used herein, “free radical damage” means the alteration in structure, function, composition, or other properties of biological tissues, organs, cells, or constituents that result from the effect of a free radical on them. Since free radicals are highly reactive and unstable species, they will in general react with a wide variety of biological targets to damage them. For example, the oxidation of lipids (lipid peroxidation), especially of cell membrane lipid, is a well-known damaging effect of radicals in biological systems.

Active Agent

The subject invention involves a method of extending the life span, in particular the average life span of mammals by reducing the level of free radicals in mammalian cells by administering to the mammal a safe and effective amount of a metal chelator selected from the group consisting of:
2-furildioxime, having the structure: 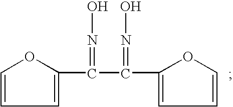
2-furilmonoxime, having the structure: 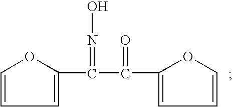
1-phenyl-1,2-propanedione-2-oxime, having the structure: 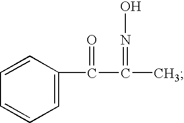
1-phenyl-1,3-butanedione (benzoylacetone), having the structure: 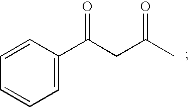
1-hydroxy-4-methyl-6-(2,4,4-trimethylpentyl)-2(1 H)-pyridinone (piroctone), having the structure: 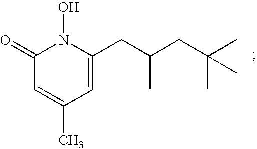
diethyldithiocarbamic acid, having the structure: 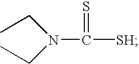
deferoxamine, having the structure: 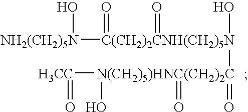
1,2-dimethyl-3-hydroxy-pyrid-4-one, (chelator L1), having the structure: 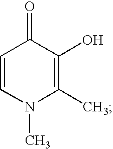
   pharmaceutically-acceptable salts thereof; and mixtures of the subject compounds and/or their salts.

Preferred active compounds of the subject invention include 2-furildioxime, 2-furilmonoxime, 1-phenyl-1,2-propanedione-2-oxime (PPDO), piroctone and diethyl-dithio-carbamic acid, and combinations thereof. More preferred compounds include 2-furildioxime, 2-furilmonoxime, piroctone and diethyldithiocarbamic acid, and combinations thereof. Even more preferred compounds include 2-furildioxime, 2-furilmonoxime, and combinations thereof. The most preferred compound of the subject invention is 2-furildioxime.

A particularly preferred piroctone salt is its ethanolamine salt, octopirox.

A particularly preferred salt of diethyldithiocarbamic acid is its sodium salt.

A particularly preferred salt of deferoxamine is its methanesulfonate salt, desferal.

Pharmaceutically-Acceptable Carrier

In addition to the active agent as described hereinbefore, the pharmaceutical compositions of the present invention essentially comprise a pharmaceutically-acceptable carrier. The term “pharmaceutically-acceptable carrier,” as used herein, means one or more compatible solid or liquid filler diluents or encapsulating substances which are suitable for administration to a human or lower animal. The term “compatible”, as used herein, means that the components of the pharmaceutical compositions are capable of being commingled with the active agent of the present invention, and with each other, in a manner such that there is no interaction which would substantially reduce the pharmaceutical efficacy of the pharmaceutical composition under ordinary use situations. Pharmaceutically-acceptable carriers must, of course, be of sufficiently high purity and sufficiently low toxicity to render them suitable for administration to the human or lower animal being treated.

Some examples of substances which can serve as pharmaceutically-acceptable carriers are sugars such as lactose, glucose and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such a peanut oil, cottonseed oil, sesame oil, olive oil, corn oil and oil of theobroma; polyols such as propylene glycol, glycerine, sorbitol, mannitol, and polyethylene glycol; sugar; alginic acid; pyrogen-free water; isotonic saline; phosphate buffer solutions; cocoa butter (suppository base); emulsifiers, such as the Tweens®; as well as other non-toxic compatible substances used in pharmaceutical formulations. Wetting agents and lubricants such as sodium lauryl sulfate, as well as coloring agents, flavoring agents, excipients, tableting agents, stabilizers, antioxidants, and preservatives, can also be present.

The choice of a pharmaceutically-acceptable carrier to be used in conjunction with the compounds of the present invention is basically determined by the way the compound is to be administered. The preferred modes of administering the compounds of the present invention are orally, topically and by injection. Suitable pharmaceutically-acceptable carriers for topical application include those suited for use in creams, gels, tapes and the like; and for oral administration include those suited for tablets and capsules.

The pharmaceutically-acceptable carrier employed in conjunction with the compounds of the present invention is used at a concentration sufficient to provide a practical size to dosage relationship. The pharmaceutically-acceptable carriers, in total, may comprise from about 0.1% to about 99.99% by weight of the pharmaceutical compositions of the present invention, preferably from about 80% to about 99.9%, more preferably from about 90% to about 99.0%, even more preferably from about 92% to about 97%, also preferably from about 94% to about 96%.

Pharmaceutically-acceptable carriers suitable for the preparation of unit dosage forms for oral administration and injection, and dosage forms for topical application are well-known in the art. Their selection will depend on secondary considerations like taste, cost, and/or shelf stability, which are not critical for the purposes of the subject invention, and can be made without difficulty by a person skilled in the art. Pharmaceutically-acceptable carriers useful in the compositions of the subject invention are described more fully hereinafter.

A. Oral Dose Forms:

Various oral dosage forms can be used, including such solid forms as tablets, capsules, granules, bulk powders and microcapsules of the drug. These oral forms comprise a safe and effective amount, usually at least about 5%, and preferably from about 10% to about 50% of the compound of the subject invention. Tablets can be compressed, enteric-coated, sugar-coated or film-coated containing suitable binders, lubricants, surfactants, diluents, disintegrating agents, coloring agents, flavoring agents, preservatives, flow-inducing agents, and melting agents. Liquid oral dosage forms include aqueous and nonaqueous solutions, emulsions, suspensions, solutions and/or suspensions reconstituted from non-effervescent granules, containing suitable solvents, preservatives, emulsifying agents, suspending agents, diluents, sweeteners, melting agents, coloring agents, and flavoring agents. Preferred carriers for oral administration include gelatin and propylene glycol. Specific examples of pharmaceutically-acceptable carriers and excipients that may be used in formulating oral dosage forms containing compounds of the subject invention are described in U.S. Pat. No. 3,903,297, Robert, issued Sep. 2, 1975, incorporated by reference herein. Techniques and compositions for making solid oral dosage forms are described in Marshall, “Solid Oral Dosage Forms,” Modern Pharmaceutics, Vol. 7. (Banker and Rhodes, editors), 359-427 (1979), incorporated herein by reference. Techniques and compositions for making tablets (compressed, formulas and molded), capsules (hard and soft gelatin) and pills are described in Remington's Pharmaceutical Sciences (Arthur Osol, editor), 1553-1593 (1980), incorporated herein by reference.

The preferred unit dosage form for oral administration is tablets, capsules and the like, comprising a safe and effective amount of a compound of the subject invention. Preferably oral dose forms comprise from about 0.001 g to about 5 g of a compound of the subject invention, more preferably from about 0.01 g to about 2 g and most preferably from about 0.1 g to about 1 g, for a 50-70 kg person.

B. Topical Dose Forms:

The compositions of the subject invention can also be administered topically to a biological subject, i.e., by the direct laying on or spreading of the composition on the skin.

The topical compositions useful in the subject invention involve compositions suitable for topical application to mammalian skin, the composition comprising a safe and effective amount of an active agent or mixture of such actives as described hereinabove, and a pharmaceutically-acceptable topical carrier. The subject compositions preferably contain from about 0.001% to about 20%, preferably from about 0.01% to about 10%, more preferably from about 0.1% to about 5%.

The topical compositions useful in the subject invention may be made into a wide variety of product types. These include, but are not limited to lotions, creams, beach oils, gels, sticks, sprays, ointments, pastes, mousses, cosmetics, shampoos, cream rinses, hair tonics and hair conditioners. These product types may comprise several types of carrier systems including, but not limited to solutions, emulsions, gels, solids, and liposomes.

The topical compositions useful in the subject invention may include a safe and effective amount of penetration enhancing agent. A preferred amount of penetration enhancing agent is from about 1% to about 5% of the composition. Examples of useful penetration enhancers, among others, are disclosed in U.S. Pat. No. 4,537,776, Cooper, issued Aug. 27, 1985; U.S. Pat. No. 4,552,872, Cooper et al., issued Nov. 12, 1985; U.S. Pat. No. 4,557,934, Cooper, issued Dec. 10, 1985; U.S. Pat. No. 4,130,667, Smith, issued Dec. 19, 1978; U.S. Pat. No. 3,989,816, Rhaadhyaksha, issued Nov. 2, 1976; U.S. Pat. No. 4,017,641, DiGiulio, issued Apr. 12, 1977; and U.S. Pat. No. 4,954,487, Cooper, Loomans & Wickett, issued Sep. 4, 1990. Other conventional skin care product additives may also be included in the compositions useful in the subject invention. For example, collagen, hyaluronic acid, elastin, hydrolysates, primrose oil, jojoba oil, epidermal growth factor, soybean saponins, mucopolysaccharides, and mixtures thereof may be used.

Various vitamins may also be included in the compositions useful in the subject invention. For example, Vitamin A, and derivatives thereof, Vitamin B₂, biotin, pantothenic, Vitamin D, and mixtures thereof may be used.

C. Injectable Dose Forms

The compounds of the subject invention are also useful when injected. The dosage of the compound of the subject invention which is both safe and effective will vary with the particular condition being treated, the severity of the condition, the duration of treatment, the specific compound employed and its usage concentration, and like factors within the specific knowledge and expertise of the attending physician and commensurate with a reasonable benefit/risk ratio associated with the use of any drug compound. The injectable dosages and dosage ranges given herein are based on delivery of the compound of the subject invention to a 70 kg human and can be adjusted to provide equivalent dosages for patients of different body weights.

Methods and materials for manufacturing injectables can be found in Remington's Pharmaceutical Sciences, 17ed., 1985, Chapter 85, p. 1518, the disclosures of which are incorporated herein by reference in their entirety. The injectable dosage forms typically contain from about 0.001 mg/ml to about 100 mg/ml, preferably from about 0.01 mg/ml to about 10 mg/ml, more preferably from about 0.1 mg/ml to about 3.0 mg/ml, of the compound of the subject invention. The injectable dosage forms are typically administered from about once a week to about four times daily, more preferably from about twice a week to about three times daily, more preferably still, about three times a week to about twice daily, also preferably about once daily. Typically, from about I ml to about 100 mls of the composition are injected, preferably from about 10 mls to about 50 mls, more preferably about 25 mls.

Combination Actives

A. Sunscreens and Sunblocks

Reduction of the level of free radicals in mammalian cells can be achieved by using combinations of the active agents together with sunscreens or sunblocks. A known inducer of free radicals is ultraviolet radiation. Thus, in topical compositions, the inclusion of sunscreens/sunblocks would increase protection against radical production and subsequent damage. Useful sunblocks include, for example, zinc oxide and titanium dioxide. The combination of an active agent, with a UVA and/or UVB sunscreen is desirable. The inclusion of sunscreens in compositions useful in the subject invention at low levels does not greatly reduce the tanning response of the user but enhances the effectiveness of the subject compositions. A wide variety of conventional sunscreening agents are suitable for use in combination with a subject active agent. Sagarin, et al., at Chapter VIII, pages 189 et seq., of Cosmetics Science and Technology disclose numerous suitable agents.

A safe and effective amount of sunscreen may be used in the compositions useful in the subject invention. The sunscreening agent must be compatible with the active agent. The composition preferably comprises from about 1% to about 20%, more preferably from about 2% to about 10% of a sunscreening agent. Exact amounts will vary depending upon the sunscreen chosen and the desired Sun Protection Factor.

B. Anti-Inflammatory Agents

In a preferred composition useful in the subject invention, an anti-inflammatory agent is included as an active along with the active agent. The inclusion of an anti-inflammatory agent enhances the benefits of the compositions because the mammalian body will respond to radical damage by mounting an inflammation response which can lead to additional cell damage. The anti-inflammatory agent also protects strongly in the UVA radiation range (though it also provides some UVB protection as well). (See U.S. Pat. No. 4,847,071, Bissett, Bush, and Chatterjee, issued Jul. 11, 1989, incorporated herein by reference; and U.S. Pat. No. 4,847,069, Bissett and Chatterjee, issued Jul. 11, 1989, incorporated herein by reference.)

A safe and effective amount of an anti-inflammatory agent may be added to the compositions useful in the subject invention, preferably from about 0.1% to about 10%, more preferably from about 0.5% to about 5%, of the composition. The exact amount of anti-inflammatory agent to be used in the compositions will depend on the particular anti-inflammatory agent utilized since such agents vary widely in potency.

C. Anti-Oxidants/Radical Scavengers

In a preferred composition useful in the subject invention, an anti-oxidant/radical scavenger is included as an active along with a subject active agent. The inclusion of an anti-oxidant/radical scavenger increases the benefits of the composition because an anti-oxidant/radcial scavenger can neutralize any free radicals that are produced in spite of the subject actives' chelating action.

A safe and effective amount of an anti-oxidant/radical scavenger may be added to the compositions useful in the subject invention, preferably from about 0.1% to about 10%, more preferably from about 1% to about 5%, of the composition.

Anti-oxidants/radical scavengers such as ascorbic acid (vitamin C) and its salts, ascorbyl phosphate and its salts (e.g., magnesium and sodium salts), tocopherol (vitamin E), tocopherol sorbate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, lipoic acid, nitro-arginine, uric acid and its salts and alkyl esters, sorbic acid and its salts, the ascorbyl esters of fatty acids, amines (e.g., N,N-diethylhydroxylamine, amino-guanidine), sulfhydryl compounds (e.g., glutathione, N-acetyl cysteine), bioflavonoids, niacinamide, and dihydroxy fumaric acid and its salts may be used.

In a preferred composition useful in the subject invention, compositions comprise one, any two, or all three of a sunscreening agent, anti-inflammatory agent, and/or an anti-oxidant/radical scavenging agent included as actives along with a subject active agent. The inclusion of two or all three of these agents with the subject active increases the benefits of the composition.

D. Chelators

In a preferred composition useful in the subject invention, an additional chelating agent is included as an active along with a subject active agent. As used herein, “chelating agent” means an active agent capable of removing a metal ion from a system by forming a complex so that the metal ion cannot readily participate in or catalyze chemical reactions. The inclusion of an additional chelating agent increases the benefits of the composition.

A safe and effective amount of a chelating agent may be added to the compositions useful in the subject invention, preferably from about 0.1% to about 10%, more preferably from about 1% to about 5%, of the composition. Chelators useful in compositions are disclosed in U.S. patent application Ser. No. 619,805, Bissett, Bush & Chatterjee, filed Nov. 27, 1990 (which is a continuation of U.S. patent application Ser. No. 251,910, filed Oct. 4, 1988); U.S. patent application Ser. No. 514,892, Bush & Bissett, filed Apr. 26, 1990; U.S. patent application Ser. No. 657,847, Bush, Bissett & Chatterjee, filed Feb. 25, 1991; and U.S. patent application Ser. No. 776,506, Bush, filed Oct. 11, 1991; all incorporated herein by reference. The additional chelators preferred include:

kojic acid
2,3-bis-(2-pyridyl)-pyrazine
3-(4-phenyl-2-pyridyl)-5-phenyl-1,2,4-triazine
2,3-bis-(2-pyridyl)-5,6-dihydropyrazine
2,4,6-tri-(2-pyridyl)-1,3,5-triazine
1-pyrrolidine carbodithioic acid
di-2-pyridyl ketone
phenyl 2-pyridyl ketoxime
2,3-dihydroxy naphthalene
2,3-dihydroxy pyrridine
3-hydroxy-2-methyl-4-pyrone
2,3-dihydroxy benzoic acid
ethylenediamine-N,N-bis-(2-hydroxy-phenylacetic acid) dimethyl ester

In a preferred composition useful in the subject invention, compositions comprise one, any two, any three, or all four of a sunscreening agent, anti-inflammatory agent, anti-oxidant/radical scavenging agent, and/or chelating agent included as actives along with the subject active agent. The inclusion of two, three, or all four of these agents with the active agent increases the benefits of the composition.

Methods of Extending the Average Lifespan of Mammals by Reducing Free Radical Damage in Mammalian Cells

The subject invention relates to methods of extending the life span, in particular the average life span of mammals by reducing free radical damage in cells of living mammals. The reduction of the level of free radicals in mammalian cells reduces the level of free radical aging in the cells.

The subject methods comprise the step of administering to a mammal a composition comprising a safe and effective amount of a metal chelator, in particular an iron chelator selected from the group consisting of 2-furildioxime, 2-furilmonoxime, 1-phenyl-1,2-propanedione-2-oxime, benzoylacetone, piroctone, diethyldithiocarbamic acid, deferoxamine and 1,2-dimethyl-3-hydroxy-pyrid-4-one, and pharmaceutically-acceptable salts thereof. The amount of treatment and frequency of application will vary widely depending upon the conditions of the cells already in existence in the subject and the level of treatment desired.

A safe and effective amount of a metal chelator, in particular an iron chelator in a topical composition, is applied, generally from about 0.001 mg to about 2 mg per cm² skin per application, preferably from about 0.005 mg to about 1 mg per cm² skin per application, more preferably from about 0.01 mg to about 0.5 mg/cm², also preferably from about 0.02 mg to about 0.2 mg/cm². Application preferably ranges from about weekly to about 5 times daily, more preferably from about twice a week to about four times daily, more preferably still from about every other day to about 3 times daily, also preferably from about once a day to about twice a day. The subject compositions are preferably applied to an area of from about 10 cm² to about 10,000 cm² skin for each application, more preferably from about 100 cm² to about 5,000 cm² skin, also preferably from about 500 cm² to about 1000 cm² skin. Treatment is continued for at least 7 days, more preferably 6 months, even more preferably 1 year, more preferably still 5 years, also preferably 10 years.

A preferred method of the subject invention involves applying both a safe and effective amount of a metal chelator, in particular an iron chelator and a safe and effective amount of one or more of a sunscreening agent, an anti-inflammatory agent, an anti-oxidant/radical scavenging agent, and/or a chelating agent, to the skin simultaneously. As used herein, “simultaneous application” or “simultaneously” means applying the agents to the skin at the same situs on the body at about the same time. Though this can be accomplished by applying the agents separately to the skin, preferably a composition comprising all the desired agents commingled is applied to the skin. The amount of sunscreening agent applied is preferably from about 0.05 mg to about 0.5 mg per cm² skin. The amount of anti-inflammatory agent applied is preferably from about 0.005 mg to about 0.5 mg, more preferably from about 0.01 mg to about 0.1 mg per cm² skin. The amount of anti-oxidant/radical scavenging agent preferably applied is from about 0.01 mg to about 1.0 mg, more preferably from about 0.05 mg to about 0.5 mg per cm² skin. The amount of chelating agent preferably applied is from about 0.001 mg to about 1.0 mg, more preferably from about 0.01 mg to about 0.5 mg, still more preferably from about 0.05 mg to about 0.1 mg per cm² skin. The amount of the active compound of the subject invention applied is preferably from about 0.001 mg to about 2 mg per cm² skin per application, more preferably from about 0.01 mg to about 1 mg per cm² skin per application.

The preferred modes of administration are orally, topically, inhalation, and parenterally (for example, by subcutaneous injection, intramuscular injection, intra-articular injection, intravenous injection and the like). Thus, specific modes of administration include, without limitation, oral, transdermal, mucosal, sublingual, intramuscular, intravenous, intraperitoneal, and subcutaneous administration, as well as topical application. Oral administration is more preferred.

Oral administration can be used to extend the life span, in particular the average life span of mammals by reducing the level of free radicals through oral dosing of a pharmaceutical composition comprised of a safe and effective amount of the metal chelator of the subject invention in a suitable oral pharmaceutical carrier. The chelator is absorbed by the gastrointestinal tract. The pharmaceutical composition may consist of solid dosage forms such as tablets, hard gelatin capsules, soft gelatin capsules, bulk powders, microcapsules, and the like of the drug. Alternately, it may consist of a liquid dosage form such as an aqueous or nonaqueous solution, emulsion, or suspension. In certain preferred embodiments, the pharmaceutical composition may also be incorporated into a food or beverage product for oral administration to a mammal, e.g., sports drink, fortified cereals, pet foods, etc.

The amount of the compound ingested depends upon the bioavailability of the compound from the oral pharmaceutical composition. Typically, however, the compounds of the subject invention are dosed in an amount of from about 0.001 mg/kg of body weight to about 1000 mg/kg, preferably from about 0.01 to about 500 mg/kg of body weight, more preferably from about 0.1 to about 200 mg/kg of body weight, also preferably from about 1 mg to about 100 mg/kg of body weight. The amount of the pharmaceutical composition depends upon the percent of compound within its formula, which is a function of the amount of the compound required per dose, its stability, release characteristics and other pharmaceutical parameters. Generally, the oral pharmaceutical composition should comprise from about 0.005% to about 50% of the compound of the subject invention.

Oral administration preferably ranges from about weekly to about 5 times daily, more preferably from about twice a week to about four times daily, more preferably still from about every other day to about 3 times daily, also preferably from about once a day to about twice a day. Treatment is continued for at least 7 days, more preferably 6 months, even more preferably 1 year, more preferably still 5 years, also preferably 10 years.

The preferred method of injectable administration depends upon the solubility and the stability of the particular active being used.

### EXAMPLES

The following examples further describe and demonstrate embodiments within the scope of the subject invention. The examples are given solely for the purpose of illustration and are not to be construed as limitations of the subject invention, as many variations thereof are possible without departing from the spirit and scope of the invention.

### Oral Dose Forms

### Example I

A composition for oral administration is prepared by combining the following components:

Component:

| | | |
|---|---|---|
| 2-furildioxime (FDO) | 1 | kg |
| Sesame oil | to 4 | liters |

The FDO is suspended in the sesame oil with the aid of sonication and is packaged in soft gelatin capsules using methods known in the art. Two of the resulting capsules, each containing 250 mg of the active, are administered to a 60 kg human, daily for a period of 4 years.

### Example II

A composition for oral administration is prepared by combining the following components:

Component

| | | |
|---|---|---|
| 2-furilmonoxime (FMO) | 250 | g |
| Propylene glycol | 1800 | ml |
| Ethyl alcohol | 175 | ml |
| Distilled water | 75 | ml |
| Artificial Cherry flavor | 10 | ml |
| FD&C Red #40 | 0.2 | g |

The above ingredients are combined to produce a syrup that is packaged under sterile conditions in 6 oz. bottles. One teaspoon of this formulation is administered to a 70 kg human, weekly for a period of one year.

### Topical Dose Forms

### Example III

A topical composition is prepared by combining the following components utilizing conventional mixing techniques.

| Component | % Weight |
|---|---|
| Ethanol: propylene glycol: water (1:1:2) | 98.7 |
| Diethyldithiocarbamic acid(DEDCA), | 1.3 |
| sodium salt | |

In a suitable vessel, the Na-DEDCA is dissolved in the ethanol:propylene glycol:water with stirring. Use of an amount of the composition to deposit about 0.02 mg/cm² of the active agent to about 200 cm² of skin is appropriate. The composition is applied twice daily, for a period of five years.

### Example IV

A topical composition is prepared by combining the following components utilizing conventional mixing techniques.

| Component | % Weight |
|---|---|
| Ethanol | 99.87 |
| PPDO | 0.13 |

In a suitable vessel, the PPDO is dissolved in the ethanol with stirring. Use of an amount of the composition to deposit about 0.001 mg/cm² of the active agent to the skin is appropriate. The composition is applied four times daily over a 1000 cm² area of skin for a period of six months.

### Example V

A topical composition is prepared by combining the following components utilizing conventional mixing techniques.

| Component | % Weight |
|---|---|
| Ethanol | 49.00 |
| Propylene glycol | 25.00 |
| Deionized water | 25.00 |
| Octopirox | 1.00 |

In a suitable vessel, the octopirox is dissolved in the ethanol with stirring. Propylene glycol and deionized water are added with stirring. Use of an amount of the composition to deposit about 0.02 mg/cm² of the active agent to 2000 cm² of skin is appropriate. The composition is applied once a week for one year.

### Example VI

A nonionic oil-in-water emulsion is prepared by combining the following components utilizing conventional mixing techniques:

| Component | % Weight |
|---|---|
| Deionized Water | 79.73 |
| Propylene Glycol | 3.00 |
| Octyl Methoxycinnamate | 7.50 |
| Cetyl Alcohol | 2.50 |
| Stearyl Alcohol | 2.50 |
| Laureth 23 | 2.00 |
| C₁₂₋₁₅ Alcohols Benzoate | 2.00 |
| EDTA | 0.37 |
| Methylparaben | 0.20 |
| Propylparaben | 0.10 |
| Deferoxamine | 0.10 |

Use of an amount of the composition sufficient to deposit about 0.004 mg/cm² skin of the active agent is appropriate. The composition is applied twice a week to about 500 cm² of skin for two years.

### Example VII

A nonionic oil-in-water emulsion is prepared by combining the following components utilizing conventional mixing techniques:

| Component | % Weight |
|---|---|
| Deionized Water | 78.73 |
| Propylene Glycol | 3.00 |
| Octyl Methoxycinnamate | 7.50 |
| Cetyl Alcohol | 2.50 |
| Stearyl Alcohol | 2.50 |
| Laureth 23 | 2.00 |
| C₁₂₋₁₅ Alcohols Benzoate | 2.00 |
| EDTA | 0.37 |
| Methylparaben | 0.20 |
| Propylparaben | 0.10 |
| Benzoylacetone | 1.10 |

Use of an amount of the composition sufficient to deposit about 0.05 mg/cm² of the active agent to about 100 cm² of skin is appropriate. The composition is applied once daily, for three years.

### Example VIII

An ion pair oil-in-water emulsion is prepared by combining the following components utilizing conventional mixing techniques.

| Component | % Weight |
|---|---|
| Deionized Water | 78.05 |
| Permulon TR-2 (C10-C30 Acrylate | 0.30 |
| Copolymer, B.F. Goodrich) | |
| Distearyl Dimethyl Ammonium Chloride | 0.15 |
| 1,2-dimethyl-3-hydroxy-pyrid-4-one | 1.00 |
| 4-N,N-(2-ethylhexyl)methylaminobenzoic acid | 4.00 |
| 4-ester of 2-hydroxy-4-(2-hydroxyethoxy)- | |
| benzophenone | |
| 4-N,N-(2-ethylhexyl)methylaminobenzoic acid | 2.00 |
| ester of 4-(2-hydroxyethoxy)dibenzoylmethane | |
| Dimethyl Isosorbide | 6.00 |
| Dioctyl Malate | 6.00 |
| Cetyl Alcohol | 1.00 |
| Stearyl Alcohol | 1.00 |
| 99% Triethanolamine | 0.50 |

Use of an amount of the composition sufficient to deposit about 0.1 mg/cm² of the active agent to about 1000 cm² of skin is appropriate. The composition is applied twice daily, for one year.

### Example IX

A sunscreen composition is prepared by combining the following components utilizing conventional mixing techniques.

| Component | % Weight |
|---|---|
| Polypropylene Glycol 15 Stearyl Ether | 15.00 |
| Sorbitan Oleate | 2.00 |
| Octyl Methoxy Cinnamate | 7.50 |
| FDO | 0.50 |
| Propyl Paraben | 0.15 |
| Butylated Hydroxy Toluene | 0.05 |
| Cyclomethicone | 20.00 |
| Sesame Oil | 5.00 |
| Mineral Oil (Blandol) | 49.80 |

Use of an amount of the composition is sufficient to deposit about 0.1 mg/cm² of the active agent to the skin is appropriate. This composition is applied three times daily, for six months.

### Experimental Data

### Example X

This experiment demonstrates the extension in the average life span of a mammal via the methods of the present invention.

Experimental Procedure

Preliminary Data Gathering:

Three key parameters were measured prior to starting the study: (a) FDO stability under the diet making conditions (70° C. for 3 hours under vacuum), (b) FDO stability to stomach pH (approximately pH 1), and (c) FDO absorbed intact into the blood stream of the mouse. The following were the outcomes:
(a) The FDO was mixed with ground Purina Mouse Chow® and heated to 70° C. for 3 hours under vacuum; the FDO was then extracted from the chow with ethanol and assayed by HPLC and by UV absorption spectrum. There was no detectable loss of FDO.
(b) The FDO was dissolved in 2:1:1 ethanol:propylene glycol:water at pH 1 (HCl); the FDO was then analyzed by HPLC and by UV absorption (obtaining the absorption spectrum vs. FDO standard and then determining level of FDO at the peak absorption). There was no detectable loss of FDO.
(c) A certain number of Skh mice were fed ground Purina Mouse Chow® containing 0.01% ¹⁴C-FDO for 14 days; blood was then withdrawn and analyzed for FDO by HPLC. Intact FDO was detected in the blood samples.
   Experiment:

Forty eight (48) female albino Skh:HR-1 hairless mice were obtained from Charles River Laboratories (Portage, Mich.). At the start of the study, the mice were approximately 20 weeks old. The mice were housed four to a standard plastic cage and given feed and water ad libitum. Sixteen (16) mice were placed on each of the three test diets described below.

The 2-Furildioxime (FDO) was obtained from Aldrich Chemical Co. (Milwaukee, Wis.). Based on HPLC analysis, the FDO sample had an isomer ratio of approximately 1:1 anti:amphi, with <1% syn. ¹⁴C-FDO was obtained from Amersham (Arlington Heights, Ill.); it had a similar isomer ratio to the unlabeled material.

The FDO-containing and control diets were prepared by Teklad (Madison, Wis.) by addition of FDO to standard Purina Mouse Chow®. Diets were stored at 4° C. in sealed containers until used. The levels of FDO in the diets were 0%, 0.01%, and 0.1%.

At 3 month intervals throughout the study, samples of the FDO-containing Purina Mouse Chow® were ground and extracted with ethanol; the ethanol was then analyzed for FDO content by UV absorption (obtaining the absorption spectrum versus the FDO standard and then determining level of FDO at the peak absorption). There was no detectable loss of FDO from the diets over the approximately two-year course of the study.

Throughout the study, mouse weights, food consumption, and date of death were recorded. Also, mice were observed for health (observation of appearance, behavior, and general health), and where possible, cause of death was determined at autopsy by a veterinarian.

Results

Mouse food consumption and body weights were not different among the three test diet groups. This indicates the mice being fed the two FDO diets were thriving relative to the no-FDO control diet. Additionally, the mice were observed on a daily basis for appearance or behaviorial differences. No differences among groups were noted.

Date of death for the mice was recorded. The data were converted to average life span (Table 1). Statistical analysis indicated both FDO diets significantly increased life span relative to the control diet.

TABLE I
Effect of dietary FDO on mouse average life span (survival)

| | Mean survival | % Increase in | Significance |
|---|---|---|---|
| Diet | (days) | Survival Time | (vs. control) |
| 0% FDO | 553 | — | — |
| 0.01% FDO | 632 | 14 | p = 0.05 |
| 0.1% FDO | 664 | 20 | p = 0.02 |

Cause of death was also determined and were one of five causes: malignant lymphoma, chronic liver inflammation, cystic ovaries, kidney amyloidosis, and chronic lung inflammation. There were not any differences in the causes of death among the diet groups. For the FDO-fed mice, death from all causes was simply delayed to a later date.

Discussion

The results indicate that oral FDO can increase the average life span of mice and would be expected to provide the same effect in other mammals. Since FDO is a metal chelator, this suggests that a metal (e.g., iron) is an important factor in chronological aging. Without being limited by theory, it is believed that the iron likely exerted its effect via production of damaging oxygen radicals. By preventing formation of damaging oxygen radicals, FDO prevents radical-induced chronological aging changes. The other chelators of the subject invention would be expected to provide a similar benefit.

While particular embodiments of the subject invention have been described, it will be obvious to those skilled in the art that various changes and modifications of the subject invention can be made without departing from the spirit and scope of the invention. It is intended to cover, in the appended claims, all such modifications that are within the scope of the invention.

1. A method of extending the life span of mammals by reducing free radical damage in cells of living mammals wherein said method comprises the step of administering to a mammal a composition comprising a safe and effective amount of an iron chelator selected from the group consisting of 2-furildioxime, 2-furilmonoxime, 1-phenyl-1,2-propanedione-2-oxime, benzoylacetone, piroctone, diethyldithiocarbamic acid, deferoxamine and 1,2-dimethyl-3-hydroxy-pyrid-4-one, their pharmaceutically-acceptable salts, and combinations thereof, wherein said composition is administered in an oral dose form, wherein from about 0.001 mg to about 1 g of the chelator is administered per kg of body weight of a mammal, from about once a week to about 5 times daily.

2. The method of claim 1 wherein said iron chelator is selected from the group consisting of 2-furildioxime, 2-furilmonoxime, 1-phenyl-1,2-propanedione-2-oxime, piroctone and diethyldithiocarbamic acid, and combinations thereof.

3. The method of claim 1 wherein said iron chelator is selected from the group consisting of 2-furildioxime, 2-furilmonoxime, piroctone and diethyldithiocarbamic acid, and combinations thereof.

4. The method of claim 1 wherein said iron chelator is selected from the group consisting of 2-furildioxime, 2-furilmonoxime, and combinations thereof.

5. The method of claim 1 wherein said iron chelator is 2-furildioxime.

6. The method of claim 1 wherein said composition is administered in an oral dose form, wherein from about 0.01 mg to about 500 mg of said chelator is administered per kg of body weight of a human, from about twice a week to about four times daily.

7. The method of claim 1 wherein said composition is administered in an oral dose form, wherein from about 0.1 mg to about 200 mg of said chelator is administered per kg of body weight of a human, from about once a day to about twice a day.

8. The method of claim 1 wherein said composition is administered for a period of ten years or more.

9. A method of extending the life span of mammals by reducing free radical damage in cells of living mammals wherein said method comprises the step of administering to a mammal a composition comprising a safe and effective amount of an iron chelator selected from the group consisting of 2-furildioxime, 2-furilmonoxime, 1-phenyl-1,2-propanedione-2-oxime, benzoylacetone, piroctone, diethyldithiocarbamic acid, deferoxamine and 1,2-dimethyl-3-hydroxy-pyrid-4-one, their pharmaceutically-acceptable salts, and combinations thereof, wherein said composition is applied topically to the skin of the mammal wherein the amount of chelator applied to the skin is from about 0.001 mg to about 2 mg per cm² skin, to an area of about 10 cm² to about 10,000 cm² of skin for each application, from about once a week to about 5 times daily.

10. The method of claim 9 wherein said iron chelator is selected from the group consisting of 2-furildioxime, 2-furilmonoxime, 1-phenyl-1,2-propanedione-2-oxime, piroctone and diethyldithiocarbamic acid, and combinations thereof.

11. The method of claim 9 wherein said iron chelator is selected from the group consisting of 2-furildioxime, 2-furilmonoxime, piroctone and diethyldithiocarbamic acid, and combinations thereof.

12. The method of claim 9 wherein said iron chelator is selected from the group consisting of 2-furildioxime, 2-furilmonoxime, and combinations thereof.

13. The method of claim 9 wherein said iron chelator is 2-furildioxime.

14. The method of claim 9 wherein said composition is applied topically to the skin of the mammal wherein the amount of chelator applied to the skin is from about 0.005 mg to about 1 mg per cm² skin, to an area of about 100 cm² to about 5000 cm² skin for each application, from about twice a week to about 4 times daily.

15. The method of claim 9 wherein said composition is applied topically to the skin of the mammal wherein the amount of chelator applied to the skin is from about 0.01 mg to about 0.5 mg per cm² skin, to an area of about 500 cm² to about 1000 cm² skin for each application, from about once a day to about twice a day.

16. The method of claim 9 wherein said composition is administered for a period of ten years or more.

17. A method of extending the life span of mammals by reducing free radical damage in cells of living mammals wherein said method comprises the step of administering to a mammal a composition comprising a safe and effective amount of an iron chelator selected from the group consisting of 2-furildioxime, 2-furilmonoxime, 1-phenyl-1,2-propanedione-2-oxime, benzoylacetone, piroctone, diethyldithiocarbamic acid, deferoxamine and 1,2-dimethyl-3-hydroxy-pyrid-4-one, their pharmaceutically-acceptable salts, and combinations thereof, wherein said composition is administered in an injectable does form from about once a week to about four times daily, and from about 1 ml to about 100 mls of the composition is injected.

18. The method of claim 17 wherein said composition is injected in the skin of the mammal from about twice a week to about three times daily, and from about 10 mls to about 50 mls of said composition is injected.

19. The method of claim 17 wherein said composition is injected in the skin of the mammal from about three times a week to about twice daily, and from about 25 mls of said composition is injected.
